(19) United States
(12) Patent Application Publication
(10) Pub. No.: US 2022/0183766 A1
(43) Pub. Date: Jun. 16, 2022

(54) SYSTEMS AND METHODS FOR DEFINING A WORK VOLUME

(71) Applicant: Mazor Robotics Ltd., Caesarea (IL)

(72) Inventors: Ziv Seemann, Beit Ytzhack (IL); Adi Sandelson, Givatayim (IL); Dor Kopito, Kibbutz Parod (IL); Nimrod Dori, Atlit (IL); Gal Eshed, Atlit (IL); Dany Junio, Tel Aviv-Yafo (IL); Elad Ratzabi, Bait Herut (IL)

(21) Appl. No.: 17/490,753

(22) Filed: Sep. 30, 2021

Related U.S. Application Data
(60) Provisional application No. 63/125,844, filed on Dec. 15, 2020.

(51) Int. Cl. A61B 34/20; A61B 34/30

(52) U.S. Cl. CPC A61B 34/20; A61B 2090/3979; A61B 34/30

(57) ABSTRACT

A method for determining a work volume includes receiving image information from an imaging device corresponding to an array of tracking markers fixed to a flexible mesh, the mesh placed over a patient and over at least one surgical instrument adjacent to or connected to the patient; determining a position of each tracking marker in the array of tracking markers based on the image information; defining a boundary for movement of a robotic arm based on determined tracking marker positions, such that the robotic arm does not contact the patient or the at least one surgical instrument during movement of the robotic arm; and controlling the robotic arm based on the defined boundary.

### CROSS REFERENCE TO RELATED APPLICATIONS

This application claims the benefit of U.S. Provisional Application No. 63/125,844, filed on Dec. 15, 2020, and entitled “Systems and Methods for Defining Work Volume”, which application is incorporated herein by reference in its entirety.

### FIELD

The present technology generally relates to surgical procedures, and more particularly relates to defining a work volume for a surgical procedure.

### BACKGROUND

Robotic surgery often requires restricting the movement of the robot during surgery to avoid harming the patient. Robotic surgery may be semi-autonomous, with a surgeon controlling the robot (whether directly or indirectly), or autonomous, with the robot completing the surgery without manual input.

### SUMMARY

Example aspects of the present disclosure include:

A method for determining a work volume according to at least one embodiment of the present disclosure comprises receiving, from an imaging device, image information corresponding to an array of tracking markers fixed to a flexible mesh, the mesh placed over a patient and over at least one surgical instrument adjacent to or connected to the patient; determining, based on the image information, a position of each tracking marker in the array of tracking markers; defining a boundary for movement of a robotic arm based on the determined tracking marker positions, such that the robotic arm does not contact the patient or the at least one surgical instrument during movement of the robotic arm; and controlling the robotic arm based on the defined boundary.

Any of the aspects herein, wherein each tracking marker of the array of tracking markers is secured to the flexible mesh with an adhesive.

Any of the aspects herein, wherein each tracking marker of the array of tracking markers is a reflective sphere.

Any of the aspects herein, wherein the flexible mesh is a sterile drape or a blanket.

Any of the aspects herein, wherein each tracking marker of the array of tracking markers is an infrared emitting diode (IRED).

Any of the aspects herein, wherein the flexible mesh comprises a net.

Any of the aspects herein, wherein at least one of the array of tracking markers comprises a selectively adjustable parameter.

Any of the aspects herein, wherein the selectively adjustable parameter is one of color, intensity, or frequency.

Any of the aspects herein, wherein a subset of tracking markers in the array of tracking markers comprises a unique characteristic relative to a remainder of tracking markers in the array of tracking markers, the unique characteristic indicative of a location at which the robotic arm may pass through the defined boundary.

Any of the aspects herein, wherein the first imaging device is an infrared (IR) camera, and wherein the second imaging device is a second IR camera.

Any of the aspects herein, wherein the method further comprises determining, based on the image information, an orientation of each tracking marker in the array of tracking markers.

Any of the aspects herein, wherein the flexible mesh substantially conforms to the patient and the at least one surgical instrument.

Any of the aspects herein, wherein the flexible mesh remains within three inches of an underlying surface of the patient or the at least one surgical instrument.

A system according to at least one embodiment of the present disclosure comprises a processor; and a memory storing instructions for execution by the processor that, when executed by the processor, cause the processor to receive, from a first imaging device in a first pose, first image information corresponding to a plurality of tracking devices flexibly connected to each other; receive, from a second imaging device in a second pose different than the first pose, second image information corresponding to the plurality of tracking devices; determine, based on the first image information and the second image information, a position of each tracking device in the plurality of tracking devices; define a work volume boundary based on the determined tracking device positions; and control the robotic arm based on the work volume boundary.

Any of the aspects herein, wherein the plurality of tracking devices is uniformly distributed across a first surface of a flexible drape, the flexible drape flexibly connecting the tracking devices to each other.

Any of the aspects herein, wherein each tracking device of the plurality of tracking devices is glued to the flexible drape.

Any of the aspects herein, wherein each tracking device of the plurality of tracking devices is physically secured within a net that flexibly connects the tracking devices to each other.

Any of the aspects herein, wherein a flexible sheet flexibly connects the plurality of tracking devices to each other, the flexible sheet comprising a plurality of receptacles, each receptacle configured to hold one of the plurality of tracking devices.

Any of the aspects herein, wherein each of the plurality of receptacles is a plastic sphere, and wherein each of the plastic spheres is injected with an IRED.

Any of the aspects herein, wherein the defined work volume boundary separates a first volumetric section from a second volumetric section, wherein the processor causes the robotic arm to move within the first volumetric section, and wherein the processor prevents the robot from maneuvering within the second volumetric section.

Any of the aspects herein, wherein the plurality of tracking devices is draped over a surgical site.

Any of the aspects herein, wherein the memory stores additional instructions for execution by the processor that, when executed, further cause the processor to cause a visual representation of the defined work volume boundary to be displayed on a display device.

A system according to at least one embodiment of the present disclosure comprises a processor; a first imaging device positioned in a first location and in communication with the processor; a blanket comprising a plurality of tracking markers arranged thereon; a robotic arm; and a memory storing instructions for execution by the processor that, when executed by the processor, cause the processor to receive, from the first imaging device, first image information corresponding to the plurality of tracking markers; determine, based on the first image information, a position of each tracking marker of the plurality of tracking markers; define a virtual surface based on the determined tracking marker positions; and control the robotic arm based on the defined virtual surface.

Any of the aspects herein, wherein the system further comprises a second imaging device positioned in a second location different from the first location and in communication with the processor.

Any of the aspects herein, wherein the memory stores additional instructions for execution by the processor that, when executed, further cause the processor to receive, from the second imaging device, second image information corresponding to the plurality of tracking markers.

Any of the aspects herein, wherein the position of each tracking marker of the plurality of tracking markers is determined using the second image information.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 illustrates a perspective view of a system for performing a surgery or surgical procedure in accordance with embodiments of the present disclosure;

FIG. 2 shows a block diagram of the structure of control components of a system in accordance with embodiments of the present disclosure;

FIG. 3 is a schematic view of a flexible sheet in accordance with embodiments of the present disclosure; and

FIG. 4 is a flowchart of a method according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

To accomplish the task of ensuring a successful surgical procedure, various types of boundary definition techniques may be utilized to ensure the movement of the robot does not harm the patient and/or the environment (e.g., other objects and/or devices in the vicinity of the robot). A three-dimensional (3D) scanning procedure may be used to ensure that the robot used in the surgery may move without injuring the patient. For example, a robot may be configured to perform a full 3D scan of the patient using a camera positioned within or on the robot. The 3D scan may then be used to determine the geometry associated with the patient and establish a 3D area of operation. The defined boundary may encompass and/or separate from a robotic work volume medical equipment (e.g., components, tools, and/or instruments) in addition to the patient. The medical equipment may be or include, for example, tools and/or other instruments connected to the patient (e.g., retractors, dilators, reference frames, cannulas, minimally invasive surgery towers). By ensuring that the defined boundary encompasses such equipment in addition to the patient herself or himself, a safety region in which no robot movement will occur may be separated from a robotic work volume, thus protecting against inadvertent contact of the robot with the equipment in question as well as the patient.

According to one embodiment of the present disclosure, a setup comprising two infrared cameras may be used to identify and track markers on a blanket or mesh according to embodiments of the present disclosure. According to another embodiment of the present disclosure, two infrared cameras may be used as in the previously described embodiment, and a secondary camera may additionally be used to track the two infrared cameras—each of which may be equipped with a tracker to facilitate such tracking. The tracking marker can be passive (e.g., a reflective sphere) or active (e.g., an infrared-emitting device (IRED), light emitting diode (LED)). According to yet another embodiment of the present disclosure, each infrared camera may be mounted to a robotic arm, and the robotic platform comprising the robotic arm(s) may be used to provide precise pose information for each infrared camera. Additionally, although the foregoing description references infrared cameras, some embodiments of the present disclosure utilize cameras other than infrared cameras, as well as trackers, markers, or other identifiable objects configured for use with the particular modality of camera being used.

Embodiments of the present disclosure utilize a mesh, blanket, or other object with integrated markers and that is capable of being draped over a patient and/or a surgical site. Many variations of such a mesh or other object are possible, and a particular variation may be selected based on a desired levels of accuracy needed for a particular circumstance. The mesh or other object may be, for example, a sterile drape with glued markers, a net with links configured to hold plastic spheres, or a blanket with integrated, always-on IREDs with draping. Any type of marker may be used in connection with the present disclosure, provided that the camera(s) used to identify and track the markers are able to do so. The mesh is placed on the region of interest or surgical field—which may comprise, for example, a patient and/or medical equipment connected to the patient—to define a work volume boundary. The mesh can be draped or sterile, and may be placed on the region of interest or surgical field for purposes of defining a work volume (and, correspondingly, a safety region or no-fly zone) at any point during a surgical procedure when definition of the work volume and/or the corresponding safety region is needed. In some embodiments of the present disclosure, the mesh may be removed and replaced on the patient multiple times throughout a surgery or surgical procedure. A display (e.g., any screen or other user interface, whether of a robotic system, a navigation system, or otherwise) may be used to show the work volume boundary, as detected by the cameras, to a surgeon or other user.

Embodiments of the present disclosure also include a workflow for using a mesh as described above. The workflow may include, for example, placing a reference marker on a surgical robot; placing a snapshot device on a robotic arm of the robot (without moving the robotic arm); positioning or otherwise securing any needed medical tools or other equipment in, on, and/or around the patient (e.g., placing minimally invasive surgery (MIS) towers, reference frames, retractors, cannulas, dilators, etc.); and placing the mesh on the surgical field or region of interest (which may comprise, as indicated above, the patient and/or any additional medical equipment attached to the patient or otherwise in the surgical environment. With this arrangement, the work volume boundary (and thus the work volume) may be determined, and the snapshot may be moved to a valid acquisition position to register the navigation coordinate system to the robotic coordinate system (or vice versa).

In some embodiments, a tracker or fiducial other than a snapshot device, but still visible to a camera or other imaging device (e.g. of a navigation system), may be used to determine a position of the mesh relative to the robot. Also in some embodiments, the mesh may be provided with fiducials visible to an X-ray imaging device (e.g., ceramic BBs) and arranged in a specific pattern on or within the mesh. Using navigation fiducials, a tracker linking optical fiducials to a coordinate system of the robot, and the X-ray fiducials, the steps of registration and determining the work volume could be completed simultaneously. Of course, the work volume may additionally or alternatively be determined at any time (including at multiple times) after registration is complete.

Embodiments of the present disclosure beneficially enable faster and/or more accurate determination of a permitted workspace for a robot. Embodiments of the present disclosure also beneficially enable position determination and tracking of both the robot and the permitted workspace during surgery, reducing the probability that the robot causes harm to the patient. Embodiments of the present disclosure further beneficially lower the threshold for accurate determination of a work volume than conventional systems, allowing for, among other things, greater choice in tracking marker choice.

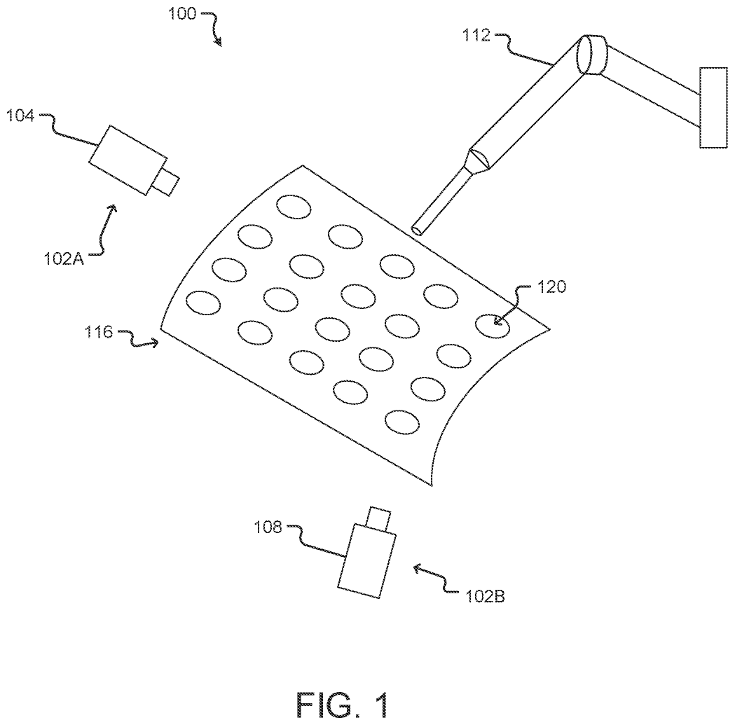

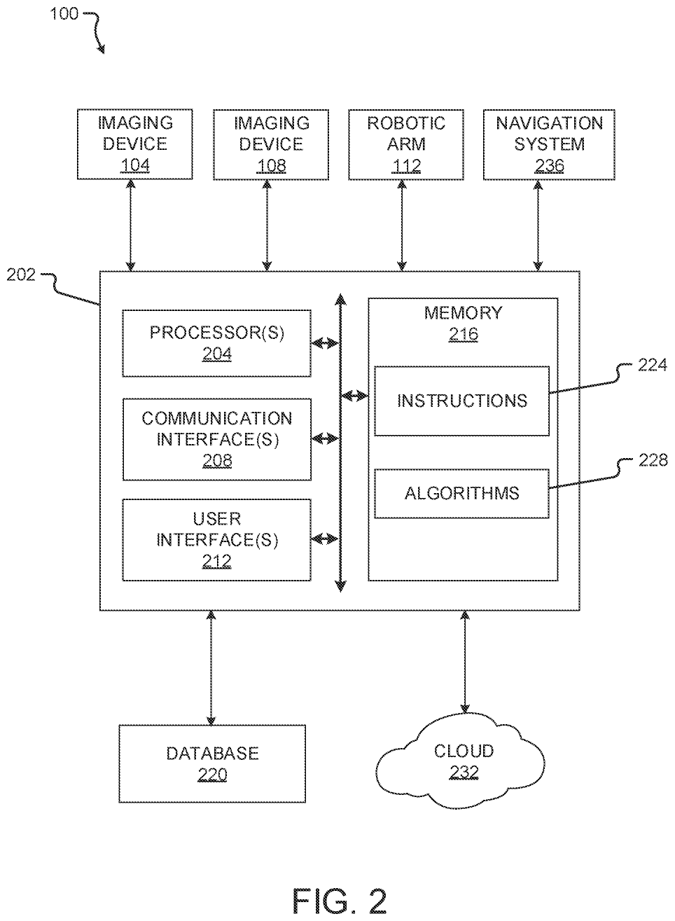

Turning first to FIGS. 1 and 2, aspects of a system 100 according to at least one embodiment of the present disclosure are shown. The system 100 may be used, for example, to determine a workspace for performing a surgery or other surgical procedure; to carry out a robotic procedure, or to gather information relevant to such a procedure; to carry out one or more aspects of one or more of the methods disclosed herein; to improve patient outcomes in connection with a robotic procedure or other surgical task or procedure; or for any other useful purpose. The system 100 includes an imaging device 104, an imaging device 108, a robotic arm 112, a mesh 116, a computing device 202, a database 220, a cloud 232, and a navigation system 236. Notwithstanding the foregoing, systems according to other embodiments of the present disclosure may omit one or more components in the system 100. For example, in some embodiments, the system 100 may omit the imaging device 108, with the imaging device 104 performing the various functions (e.g., capturing, transmitting, and/or analyzing images, image data, etc.) associated with the imaging device 108. Additionally, systems according to other embodiments of the present disclosure may arrange one or more components of the system 100 differently (e.g., the imaging devices 104, 108, the robotic arm 112, and/or the navigation system 236 may comprise one or more of the components of a computing device 202, and/or vice versa), and/or include additional components not shown.

The imaging device 104 is configured to capture, store, and/or transmit images and/or image data (e.g., image metadata, pixel data, etc.) between various components of the system 100 (e.g., to the imaging device 108, the robotic arm 112, the computing device 202, any combination thereof, etc.). The imaging device 104 may comprise one or more sensors, which may assist the system 100 in determining the position and orientation (e.g., pose) of the imaging device 104. In some embodiments, the system 100 may determine the position and orientation of the imaging device 104 relative to one or more other components (e.g., the imaging device 108, the robotic arm 112, etc.) in the system 100. The determination of the position and orientation of the imaging device 104 may assist the system 100 when processing data related images captured by the imaging device 104. For example, knowledge of the position and orientation information associated with the imaging device 104, in conjunction with other positional information (e.g., pose information related to imaging device 108) may allow one or more components of the system (e.g., the computing device 202) to determine a work volume associated with the mesh 116.

In some embodiments, the imaging device 104 comprises a one or more tracking markers attached or otherwise affixed thereto, which tracking markers are detectable by a navigation system and useful for enabling the navigation system to determine a position in space of the imaging device 104. The one or more tracking markers may be or include, for example, one or more reflective spheres, one or more IREDs, one or more LEDs, or any other suitable tracking marker. Additionally or alternatively, visual markers that are not infrared-specific may be used. For example, colored spheres, RFID tags, QR-code tags, barcodes, and/or combinations thereof may be used. In other embodiments, the imaging device 104 does not have tracking markers. In yet other embodiments, the imaging device 104 may be mounted to a robotic system, with the robotic system providing pose information for the imaging device.

The imaging device 104 is not limited to any particular imaging device, and various types of imaging devices and/or techniques may be implemented. For instance, the imaging device 104 may be capable of capturing images and/or image data across the electromagnetic spectrum (e.g., visible light, infrared light, UV light, etc.). In one embodiment, the imaging device 104 may include one or more infrared cameras (e.g., thermal imagers). In such embodiments, each infrared camera may measure, capture an image of, or otherwise determine infrared light transmitted by or from the imaged element, and may capture, store, and/or transmit the resulting information between various components of the system 100. While some embodiments of the present disclosure include infrared cameras, other embodiments may make use of other cameras and/or imaging devices. Any camera type capable of capturing images and/or image data may be used. For instance, cameras capable of capturing visible light may be used in addition to, together with, and/or instead of infrared cameras. In some embodiments, the imaging device 104 may be configured to receive one or more signals from one or more components in the system 100. For instance, the imaging device 104 may be capable of receiving one or more signals from a plurality of tracking markers 120 positioned on the mesh 116. In some embodiments, the tracking markers 120 may emit a signal (e.g., an RF signal), which the imaging device 104 may capture. The system 100 may determine (e.g., using a computing device 202) the frequencies of the RF signals, and may determine a position of each of the tracking markers 120 using the RF signals.

In the embodiment depicted in FIG. 1, the first imaging device 104 is at a first location and orientation (e.g., pose) 102A. The first pose 102A may be a point from which the imaging device 104 may view one or more of the imaging device 108, the robotic arm 112, and the mesh 116. For instance, when in the first pose 102A, the imaging device 104 may view the mesh 116 in a first orientation. In some embodiments, one or more portions of the mesh 116 may be obscured from the view of the first imaging device 104 (e.g., some tracking markers of the plurality of tracking markers 120 may be hidden from view of the first imaging device 104). In such embodiments, the first imaging device 104 may be moved to a second pose to capture additional images or other image information of the mesh 116 or portions thereof. The first imaging device 104 may be mounted to a robotic arm or to a manually adjustable mount for this purpose. Also in some embodiments, the first pose 102A may be selected to ensure that the imaging device 104 has a line of sight to an entirety of the mesh 116, or at least to each tracking marker in the plurality of tracking markers 120 on the mesh 116.

While in the first pose 102A, the imaging device 104 may be configured to capture an image (e.g., photo, picture, etc.) of the mesh 116. The captured image of the mesh 116 may depict the mesh 116 in the first orientation, with different elements of the mesh (e.g., a plurality of tracking markers) at different distances and angles relative to the imaging device 104 in the first pose 102A. In some embodiments, the imaging device 104 may then store and/or transmit the captured image to one or more components of the system 100 (e.g., the imaging device 108, the robotic arm 112, the computing device 202, the database 220, the cloud 232, and/or the navigation system 236, etc.). The same process may be repeated for any additional poses to which the imaging device 104 is moved.

The imaging device 108 is configured to capture, store, and/or transmit images and/or image data (e.g., image metadata, pixel data, etc.) between various components of the system 100 (e.g., to the imaging device 108, the robotic arm 112, combinations thereof, etc.). The imaging device 108 may be similar to, if not the same as, the imaging device 104. In some embodiments, the imaging device 108 may be disposed at a second location and orientation (e.g., pose) 102B. The second pose 102B may be a pose different from the first pose 102A, such that one or more portions of the mesh 116 may be seen by the imaging device 108 from a different view than that seen by the first imaging device 104. Even so, in some embodiments, one or more portions of the mesh 116 may be obscured from the view of the second imaging device 108 (e.g., some tracking markers of the plurality of tracking markers 120 may be hidden from view of the second imaging device 108). In such embodiments, the second imaging device 108 may be moved to a different pose to capture additional images or other image information of the mesh 116 or portions thereof. The second imaging device 108 may be mounted to a robotic arm or to a manually adjustable mount for this purpose. Also in some embodiments, the second pose 102B may be selected to ensure that the imaging device 108 has a line of sight to an entirety of the mesh 116, or at least to each tracking marker in the plurality of tracking markers 120 on the mesh 116.

While in the second pose 102B, the imaging device 108 may be configured to capture an image (e.g., photo, picture, etc.) of the mesh 116. The captured image of the mesh 116 may depict the mesh 116 in the second orientation different from the first orientation, with different elements of the blanket (e.g., different tracking markers of the plurality of tracking markers 120) at different relative distances and angles than those depicted in any images captured by the first imaging device 104 in the first pose 102A. In some embodiments, the imaging device 104 may then store and/or transmit the captured image to one or more components of the system 100 (e.g., the imaging device 108, the robotic arm 112, the computing device 202, the database 220, the cloud 232, and/or the navigation system 236, etc.). The same process may be repeated for any additional poses to which the imaging device 104 is moved.

In some embodiments, the imaging device 104 may comprise two cameras (e.g., infrared cameras) spaced apart. For instance, the first camera may be in a first pose (e.g., a first pose 102A), while the second camera may be in a second pose (e.g., a second pose 102B). In such embodiments, an imaging device 108 may or may not be utilized. The first pose and the second pose may be different from one another but may have a fixed relationship relative to one another. For example, both cameras may be mounted or otherwise attached to a frame or other structure of the imaging device 104. In some embodiments, for example, the cameras may be the cameras of a navigation system such as the navigation system 236. The positioning of the two cameras on the imaging device 104 may permit the imaging device 104 to capture three-dimensional information (e.g., in order to determine a work volume) without the need for either camera to be repositioned. In some embodiments, the system 100 may comprise additional and/or alternative cameras. In such embodiments, the imaging device 104 and/or the imaging device 108 may comprise fiducial markers (e.g., markers similar to the plurality of tracking markers 120). The additional cameras may track the fiducial markers such that the system 100 and/or components thereof may be able to determine the poses of the imaging device 104 (and/or of the cameras thereof) and/or of the imaging device 108 (e.g., the first pose 102A and/or the second pose 102B).

In some embodiments, the images captured by the imaging device 104 and/or the imaging device 108 may be used to verify a registration (e.g., a transformation of different sets of data, such as the data associated with the captured images, into a single coordinate system, or a correlation of one coordinate system or space to another coordinate system or space) for a surgery or surgical procedure. For example, the surgery or surgical procedure may comprise registering a coordinate system of a robot and/or robotic arm (e.g., a robotic arm 112), to a coordinate system of a patient. In some embodiments, a coordinate system or space of a navigation system may additionally or alternatively be registered to a robotic coordinate system and/or to a patient coordinate system. The registration may thereafter enable the robot to be moved to (and/or to avoid) specific locations relative to the patient. However, if a position of one or more of the patient, the robot, and/or the navigation system changes relative to any other one or more of the patient, the robot, and/or the navigation system, then the registration may become invalid. Images from the imaging device 104 and/or from the imaging device 108 may therefore be used to determine whether the registered entities are or are not still in the same position relative to each other.

Images captured by the imaging device 104 and/or the imaging device 108 may also be used to update a registration or to perform an additional registration, whether because the patient moved relative to the robot or vice versa or for any other reason. The system 100 and/or components thereof (e.g., a computing device 202) may then use the updated or additional registration going forward.

The robotic arm 112 may be any surgical robot arm or surgical robotic system containing a robotic arm. The robotic arm 112 may be or comprise, for example, the Mazor X™ Stealth Edition robotic guidance system. The robotic arm 112 may, in some embodiments, assist with a surgical procedure (e.g., by holding a tool in a desired trajectory or pose, by supporting the weight of a tool while a surgeon or other user operates the tool, by moving a tool to a particular pose under control of the surgeon or other user, and/or otherwise) and/or automatically carry out a surgical procedure.

The robotic arm 112 may have three, four, five, six, seven, or more degrees of freedom. The robotic arm 112 may comprise one or more segments. Each segment may be secured to at least one adjacent member by a joint, such that the robotic arm 112 is articulated. The joint(s) may be any type of joint that enables selective movement of the member relative to the structure to which the joint is attached (e.g., another segment of the robotic arm). For example, the joint may be a pivot joint, a hinge joint, a saddle joint, or a ball-and-socket joint. The joint may allow movement of the member in one dimension or in multiple dimensions, and/or along one axis or along multiple axes. While a proximal end of the robotic arm 112 may be secured to a base (whether via a joint or otherwise), a distal end of the robotic arm 112 may support an end effector. The end effector may be, for example, a tool (e.g., a drill, saw, imaging device) or a tool guide (e.g., for guiding a biopsy needle, ablation probe, or other tool along a desired trajectory).

The robotic arm 112 may comprise one or more pose sensors. The pose sensors may be configured to detect a pose of the robotic arm or portion thereof, and may be or comprise one or more rotary encoders, linear encoders, incremental encoders, or other sensors. Data from the pose sensors may be provided to a processor of the robotic arm 112, to a processor 204 of the computing device 202, and/or to the navigation system 236. The data may be used to calculate a position in space of the robotic arm 112 relative to a predetermined coordinate system. Such a calculated position may be used, for example, to determine a position in space of one or more of the plurality of sensors that are attached to the robotic arm 112. Additionally and/or alternatively, one or more tracking markers may be affixed or otherwise attached to the robotic arm 112, and the navigation system 236 may utilize the one or more tracking markers to determine a position in space (e.g., relative to a navigation coordinate system) of the robotic arm 112 and/or of an end effector supported thereby.

Embodiments of the present disclosure may comprise systems 100 with more than one robotic arm 112. For example, one or more robotic arms may be used to support one or both of the imaging devices 104 and 108. As another example, multiple robotic arms may be used to hold different tools or medical devices, each of which may need to be used simultaneous to successfully complete a surgical procedure.

The mesh 116 may be placed on (e.g., draped over, laid over, positioned on, caused to rest on) a location during a surgery or surgical procedure. For example, the mesh 116 may be draped over a patient on whom the surgery or surgical procedure is to be/being performed. The mesh 116 may also be positioned over, for example, one or more surgical instruments affixed to the patient, such as one or more retractors, minimally invasive surgery ports, cannulas, dilators, bone mount accessories used to attach a robot to one or more bones or other anatomical features of a patient, navigation markers, and/or other devices. The mesh 116 may, in some embodiments, reduce the risk of the patient being exposed to or coming into contact with hazardous material (e.g., bacteria) and may reduce the risk of surgical site infections during the surgery or surgical procedure. Embodiments of the mesh 116 may have various sizes (e.g., different dimensions in the length and width of the mesh 116) and may be designed for various surgeries or surgical tasks (e.g., spinal surgeries, laparoscopy, cardiothoracic procedures, etc.). The mesh 116 may be made of a flexible or semi-flexible material. For example, the mesh 116 may be a flexible sheet (e.g., drape, linen, etc.) made of a material that permits the mesh 116 to be deformed and/or to conform to the contours (e.g., geometry, shape, etc.) of objects over which the sheet is placed. In some embodiments, the mesh may comprise a netting or grid of rigid members that are flexibly secured to each other, such that the mesh as a whole may generally conform to the contours of any objects over which it is placed, but the individual members of the netting remain rigid. The material of the mesh 116 may include, but is in no way limited to, cotton fabrics, plastics, polypropylene, paper, combinations thereof, and/or the like.

In some embodiments, the flexible material of the mesh 116 may allow the mesh 116 to substantially conform to the surface over which the mesh 116 is placed. In particular, the mesh 116 may be sufficiently flexible to accommodate sharp transitions in the underlying geometry of the surgical field or region of interest over which the mesh is placed. In any given surgical procedures, the surgical field or region of interest over which the mesh 116 is placed may contain, in addition to anatomical surfaces, one or more medical tools or other equipment, any or all of which may extend to various lengths and at various directions. Together, these anatomical surfaces, tools, and/or equipment may comprise a number of sharp transitions (in contrast to a smooth, continuous surface). When a mesh 116 is draped over this surgical field or region of interest with its sharp transitions, the flexibility of the mesh 116 may affect how well the mesh 116 conforms to the underlying surfaces. The more inflexible the mesh 116, the more the mesh 116 will create tents (e.g., areas where the mesh 116 does not conform to the patient and/or a piece of medical equipment due sudden changes in relative height or other sharp transitions). Such tents encompass wasted space in which a robot could operate safely but is prevented from doing so due to the limitations of the mesh 116 and the resulting work volume determination. To reduce tenting, the mesh 116 may be configured (e.g., through material choice, weighted portions, etc.) to conform to the underlying geometry, including any sharp transitions, in the surgical field or region of interest.

In some embodiments, the mesh 116 may be configured to substantially conform to the underlying geometry. Unless otherwise specified, “substantially conform” as used herein means that the mesh is within one inch of an underlying surface of the surgical field or region of interest. In other embodiments, “substantially conform” may mean that the mesh is within one inch of an underlying surface, or two inches of an underlying surface, or within three inches of an underlying surface, or within four inches of an underlying surface, or within five inches of an underlying surface. In some embodiments, the mesh 116 may be flexible enough that the system 100 may be able to determine profiles of one or more components under the mesh 116 (e.g., contours of a patient, contours of medical equipment, combinations thereof, etc.) while the mesh 116 is covering the one or more components. Also in some embodiments, the system 100 can identify one or more components underneath the mesh, and their pose (whether exactly or approximately) based on the profile thereof as covered by the mesh 116 (e.g., the system 100 may compare the captured images against known profile data for each of the one or more components). In such embodiments, the system 100 may use stored information about the identified components to define the work volume, in addition to work volume boundary information based on the position of the mesh 116 itself.

The mesh 116 comprises a plurality of tracking markers 120. The plurality of tracking markers 120 may be positioned in on and/or embedded in (e.g., partially or completely) the mesh 116. The plurality of tracking markers 120 may assist the system 100 in determining one or more orientations of the mesh 116 and/or in determining a work volume (e.g., for performing a surgical procedure). For example, one or more components of the system 100 (e.g., the imaging devices 104, 108) may capture information associated with the plurality of tracking markers 120 (e.g., locations, orientations, poses, positions, etc.), and another one or more components of the system (e.g., a processor 204) may utilize the captured information to determine a position in space of the plurality of tracking markers 120 (e.g., relative to a navigation and/or a robotic coordinate system) and to determine, based on the determined position in space of the plurality of tracking markers 120, a work volume for the robot/robotic arm 112.

The density of the plurality of tracking markers 120 (e.g., the number of tracking markers 120 in a given area of the mesh 116) may change based on the type of surgery or surgical procedure and/or the number and type of medical equipment used during the surgery or surgical procedure. In embodiments where the surgical procedure includes medical equipment, the density of the plurality of tracking markers 120 may be higher, to provide a more detailed map of the working volume. In some embodiments, a required or recommended density of the plurality of tracking markers 120 may be determined by the system 100 (e.g., the system 100 may determine whether a current density of tracking markers 120 is sufficient and may alert a user if the density is insufficient to determine a working volume, or is less than recommended to accurately determine a working volume).

In some embodiments, the work volume may be determined for use in connection with manual (e.g., navigated and/or non-robotic) procedures. For example, a user (e.g., a surgeon) may use the defined work volume to perform a surgery or surgical procedure. In such embodiments, the system 100 may render the work volume to a display device (e.g., a user interface 212) to permit the user to view a virtual representation of the work volume. The system 100 may update the work volume (e.g., render an updated work volume representation to the display device) as the user performs the surgery or surgical task. Where the surgery is navigated, the navigation system may generate an alert or otherwise warn a user if a navigated tool approaches and/or crosses the determined work volume boundary.

With continued reference to FIG. 2, a block diagram of components of the system 100 according to at least one embodiment of the present disclosure is shown. These components include the imaging devices 104 and 108, the robotic arm 112, a navigation system 236, a computing system 202, a database or other data storage device 220, and a cloud or other network 232. Notwithstanding the foregoing, systems according to other embodiments of the present disclosure may omit one or more aspects of the system 100 as illustrated in FIG. 2, such as the robotic arm 112, the database 220, and/or the cloud 232. Additionally, systems according to other embodiments of the present disclosure may arrange one or more components of the system 100 differently (e.g., the imaging devices 104, 108, robotic arm 112, and/or the navigation system 236 may comprise one or more of the components of the computing device 202, and/or vice versa), and/or may include additional components not depicted in FIG. 2.

The computing device 202 comprises at least one processor 204, at least one communication interface 208, at least one user interface 212, and at least one memory 216. A computing device according to other embodiments of the present disclosure may omit one or both of the communication interface(s) 208 and/or the user interface(s) 212.

The at least one processor 204 of the computing device 202 may be any processor identified or described herein or any similar processor. The at least one processor 204 may be configured to execute instructions 224 stored in the at least one memory 216, which instructions 224 may cause the at least one processor 204 to carry out one or more computing steps utilizing or based on data received, for example, from the imaging devices 104, 108, the robotic arm 112, and/or the navigation system 236, and/or stored in the memory 216. The instructions 224 may also cause the at least one processor 204 to utilize one or more algorithms 228 stored in the memory 216. In some embodiments, the at least one processor 204 may be used to control the imaging devices 104, 108, the robotic arm 112, and/or the navigation system 236 during a surgical procedure, including during an imaging procedure or other procedure being carried out autonomously or semi-autonomously by the robotic arm 112 using the navigation system 236.

The computing device 202 may also comprise the at least one communication interface 208. The at least one communication interface 208 may be used for receiving sensor data (e.g., from the imaging devices 104 and/or 108, the robotic arm 112 and/or the navigation system 236), a surgical plan or other planning data, or other information from an external source (such as the database 220, the cloud 232, and/or a portable storage medium (e.g., a USB drive, a DVD, a CD)), and/or for transmitting instructions, images, or other information from the at least one processor 204 and/or the computing device 202 more generally to an external system or device (e.g., another computing device 202, the imaging devices 104, 108, the robotic arm 112, the navigation system 236, the database 220, the cloud 232, and/or a portable storage medium (e.g., a USB drive, a DVD, a CD)). The at least one communication interface 208 may comprise one or more wired interfaces (e.g., a USB port, an ethernet port, a Firewire port) and/or one or more wireless interfaces (configured, for example, to transmit information via one or more wireless communication protocols such as 802.11a/b/g/n, Bluetooth, Bluetooth low energy, NFC, ZigBee, and so forth). In some embodiments, the at least one communication interface 208 may be useful for enabling the device 202 to communicate with one or more other processors 204 or computing devices 202, whether to reduce the time needed to accomplish a computing-intensive task or for any other reason.

The at least one user interface 212 may be or comprise a keyboard, mouse, trackball, monitor, television, touchscreen, button, joystick, switch, lever, and/or any other device for receiving information from a user and/or for providing information to a user of the computing device 202. The at least one user interface 212 may be used, for example, to receive a user selection or other user input in connection with any step of any method described herein; to receive a user selection or other user input regarding one or more configurable settings of the computing device 202, the imaging devices 104, 108, the robotic arm 112, the navigation system 236, and/or any other component of the system 100; to receive a user selection or other user input regarding how and/or where to store and/or transfer data received, modified, and/or generated by the computing device 202; and/or to display information (e.g., text, images) and/or play a sound to a user based on data received, modified, and/or generated by the computing device 202. Notwithstanding the inclusion of the at least one user interface 212 in the system 200, the system 200 may automatically (e.g., without any input via the at least one user interface 212 or otherwise) carry out one or more, or all, of the steps of any method described herein.

Although the at least one user interface 212 is shown as part of the computing device 202, in some embodiments, the computing device 202 may utilize a user interface 212 that is housed separately from one or more remaining components of the computing device 202. In some embodiments, the user interface 212 may be located proximate one or more other components of the computing device 202, while in other embodiments, the user interface 212 may be located remotely from one or more other components of the computer device 202.

The at least one memory 216 may be or comprise RAM, DRAM, SDRAM, other solid-state memory, any memory described herein, or any other tangible non-transitory memory for storing computer-readable data and/or instructions. The at least one memory 216 may store information or data useful for completing, for example, any step of the method 400 described herein. The at least one memory 216 may store, for example, instructions 224, and/or algorithms 228. In some embodiments, the memory 216 may also store one or more preoperative and/or other surgical plans; one or more images of one or more patients, including in particular of an anatomical feature of the one or more patients on which one or more surgical procedures is/are to be performed; images and/or other data received from the imaging devices 104, 108 (or either one of the foregoing), the robotic arm 112, and/or the navigation system 236 (including any component thereof) or elsewhere; and/or other information useful in connection with the present disclosure.

The instructions 224, as described above, may be or comprise any instructions for execution by the at least one processor 204 that cause the at least one processor to carry out one or more steps of any of the methods described herein. The instructions 224 may be or comprise instructions for determining a work volume boundary based on one or more images of a mesh 116; instructions for determining a work volume based on a detected or determined work volume boundary; instructions for manipulating a robotic arm such as the robotic arm 112 to carry out a surgical procedure based on a determined work volume and/or work volume boundary; or otherwise. The instructions 224 may additionally or alternatively enable the at least one processor 204, and/or the computing device 202 more generally, to operate as a machine learning engine that receives data and outputs one or more thresholds, criteria, algorithms, and/or other parameters that can be utilized during an interbody implant insertion procedure, and/or during any other surgical procedure in which information obtained from an interbody tool as described herein may be relevant, to increase the likelihood of a positive procedural outcome.

The algorithms 228 may be or comprise any algorithms useful for converting sensor data received from sensors (including imaging sensors of the imaging devices 104, 108) and/or from gauges into meaningful information (e.g., spatial position information relative to a given coordinate system, a continuous work volume boundary, a calculated force value, a pressure value, a distance measurement). The algorithms 228 may further be or comprise algorithms useful for controlling the imaging devices 104, 108, the robotic arm 112, and/or the navigation system 236. The algorithms 228 may further be or comprise any algorithms useful for calculating whether a command for a particular movement of a robotic arm such as the robotic arm 112 will cause the robotic arm to violate a determined work volume boundary, for determining a work volume, and/or for calculating movements of a robotic arm that will maintain the robotic arm within the work volume. The algorithms 228 may further be or comprise algorithms useful for generating one or more recommendations to a surgeon or other user of the system 200 based on information received from a sensor and/or a gauge, and/or for modifying a preoperative or other surgical plan based on such information and/or an evaluation of such information. In some embodiments, the algorithms 228 may be or include machine learning algorithms useful for analyzing historical data (e.g., stored in the database 220).

The database 220 may store any information that is shown in FIG. 2 and/or described herein as being stored in the memory 216, including instructions such as the instructions 224 and/or algorithms such as the algorithms 228. In some embodiments, the database 220 stores one or more preoperative or other surgical plans. The database 220 may additionally or alternatively store, for example, information about or corresponding to one or more characteristics of one or more of the imaging device 104, the imaging device 108, the robotic arm 112, the mesh 116, and the plurality of tracking markers 120; information about one or more available mesh sizes and/or profiles, and/or other information regarding available tools and/or equipment for use in connection with a surgical procedure. The database 220 may be configured to provide any such information to the imaging devices 104, 108, the robotic arm 112, the computing device 202, the navigation system 236, or to any other device of the system 100 or external to the system 100, whether directly or via the cloud 232. In some embodiments, the database 220 may be or comprise part of a hospital image storage system, such as a picture archiving and communication system (PACS), a health information system (HIS), and/or another system for collecting, storing, managing, and/or transmitting electronic medical records including image data. Also in some embodiments, the memory 216 may store any of the information described above.

The cloud 232 may be or represent the Internet or any other wide area network. The computing device 202 may be connected to the cloud 232 via the communication interface 208, using a wired connection, a wireless connection, or both. In some embodiments, the computing device 202 may communicate with the database 220 and/or an external device (e.g., a computing device) via the cloud 232.

The navigation system 236 may provide navigation for a surgeon and/or for the robotic arm 112 during an operation or surgical procedure. The navigation system 236 may be any now-known or future-developed navigation system, including, for example, the Medtronic StealthStation™ S8 surgical navigation system. The navigation system 236 may include a camera or other sensor(s) for detecting and/or tracking one or more reference markers, navigated trackers, or other objects (e.g., a plurality of tracking markers 120) within an operating room or other room where a surgical procedure takes place. In some embodiments, the navigation system 236 may comprise the plurality of sensors. In various embodiments, the navigation system 236 may be used to track a position of one or more imaging devices 104, 108, of the robotic arm 112, and/or of one or more other objects to which the navigation system 236 has a line of sight (where the navigation system is an optical system) or that are otherwise detectable by the navigation system 236. The navigation system 236 may be used to track a position of one or more reference markers or arrays or other structures useful for detection by a camera or other sensor of the navigation system 236. The navigation system 236 may include a display for displaying one or more images from an external source (e.g., the computing device 202, the cloud 232, or other source) or a video stream from the navigation camera, or from one or both of the imaging devices 104, 108, or from another sensor. In some embodiments, the system 200 may operate without the use of the navigation system 236.

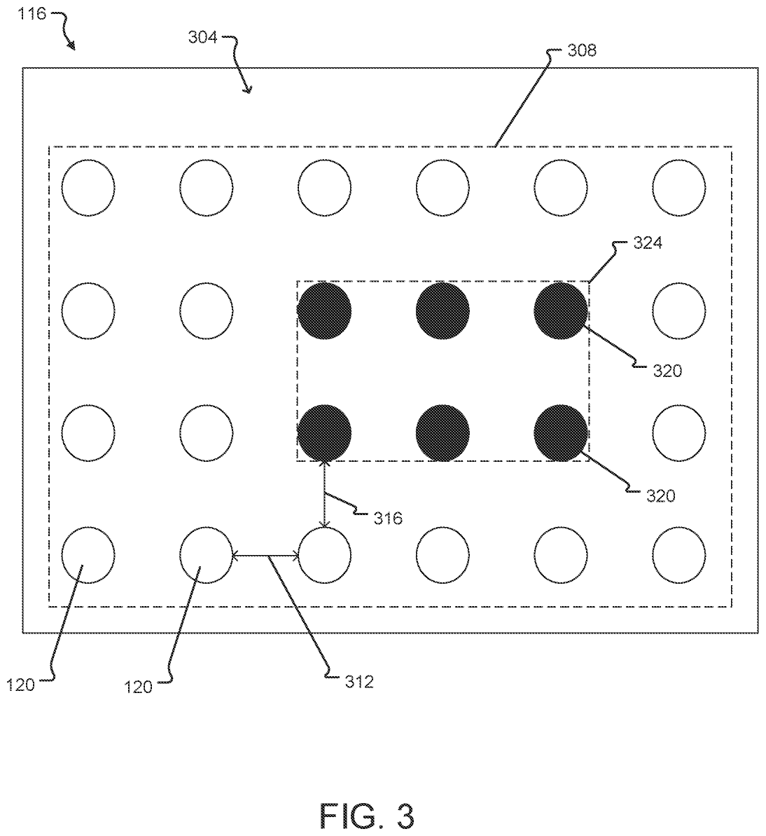

Turning to FIG. 3, a mesh 116 is shown in accordance with at least one embodiment of the present disclosure. The mesh 116 may be arranged proximate to (e.g., draped, placed over, resting on, etc.) a patient or other surgical site at any point before and/or during a surgery or surgical procedure. The mesh 116 (and more specifically, the tracking markers 120 affixed thereto) may then be imaged using the imaging devices 104, 108, after which the mesh 116 may be removed. The images generated by the imaging devices 104, 108 may be analyzed by the processor 204 or another processor to determine a position, relative to a predetermined coordinate system, of the tracking markers 120 in the images. The processor 204 or other processor then uses the determined positions of the tracking markers 120 to define a virtual surface (corresponding generally to the surface 304 of the mesh 116 when the mesh 116 was resting on the patient) that constitutes a work volume boundary 308. This work volume boundary is then used to define a work volume in which a robot (including, for example, a robotic arm such as the robotic arm 112) may safely maneuver, as well as a “no-fly zone” into which the robot will be prevented from moving (at least automatically). More specifically, the volume above the work volume boundary 308 (e.g., on an opposite side of the work volume boundary 308 from the patient) is defined as the working volume, while the volume underneath the work volume boundary (e.g., on the same side of the work volume boundary 308 as the patient) becomes the safety region or “no-fly zone.” In some embodiments (such as when a patient is in a lateral pose), the working volume may include a volume to the side of the patient.

In some embodiments, the robot may be capable of entering the no-fly zone, but only at a lower speed, with an increased sensitivity, or under manual control. For example, the movement of the robot in the no-fly zone may be constrained by physical contact. In other words the robot, when in the no-fly zone, may immediately stop upon contact with any elements or components in the no-fly zone (e.g., contact with a patient and/or other surgical instruments in the no-fly zone). In some embodiments, the robot may be directed into the no-fly zone by a user (e.g., a surgeon). In such embodiments, the user may be able to override the defined no-fly zone by issuing commands (e.g., via the user interface 212) to the robot to enter the no-fly zone.

The mesh 116 may be a flexible sheet (e.g., a sterile or non-sterile drape, depending for example on whether the surgery has begun) formed from any flexible material capable of conforming to the contours of a patient and/or any other objects upon which the mesh is arranged, as discussed above. (In some embodiments, the mesh 116 may comprise a plurality of rigid elements flexibly connected so as to enable the mesh to conform to the contours of a patient and/or any other objects upon which the mesh is arranged.) The mesh 116 comprises a first surface 304 and a plurality of tracking markers 120. The tracking markers 120 may be disposed on or partially or wholly inside of the mesh 116 (e.g., under the first surface 304). For example, the tracking markers 120 may be secured (e.g., adhered with an adhesive (e.g., glue), stitched, sewn, held in one or more pockets, any combination of the foregoing, etc.) to the first surface 304 of the mesh 116. In some embodiments, the mesh 116 may be or comprise a net. In other words, the mesh 116 may comprise the plurality of tracking markers 120, with each tracking marker of the tracking markers 120 being flexibly connected (e.g., connected by strings, lines, or the like) forming a mesh with space between each of the tracking markers 120. In such embodiments, the mesh containing the tracking markers 120 may be used independently as a mesh 116 or may be affixed to a flexible sheet or other fabric to form the mesh 116.

In some embodiments, the tracking markers 120 may be spaced apart from one another by a first distance 312 in a first direction (e.g., a horizontal direction) and/or by a second distance 316 in a second direction (e.g., a vertical distance). In some embodiments, the first distance and the second distance may be equal in value and the tracking markers 120 may be uniformly distributed across the first surface 304 of the mesh 116. The tracking markers 120 may alternatively be disposed in any known pattern or defined shape. Additionally or alternatively, the tracking markers 120 may be disposed along the boundary of the mesh 116. In some embodiments, the plurality of tracking markers 120 may be randomly distributed across the mesh 116 (e.g., the plurality of tracking markers 120 have no discernable or intentional pattern). The spacing of the tracking markers 120 may be known to one or more components of the system 100 (e.g., stored in the database 220 and capable of being accessed by the system 100), and such spacing information may be utilized by the system 100 together with images or other image information received from the imaging devices 104, 108 to determine a work volume boundary based on the detected arrangement of the mesh 116 (whether relative to a particular coordinate system and/or relative to one or both of the imaging devices 104, 108). In some embodiments, the tracking markers 120 may comprise various shapes and/or sizes and may cover various sections of the mesh 116. Examples of possible shapes of the tracking markers 120 include spherical, cylindrical, polygonal, and/or the like. The variations in shapes and/or sizes may assist any number of components of the system 100 in determining positions and/or orientations of one or more of the tracking markers 120.

In some embodiments, the tracking markers 120 may provide indicia that may assist the system 100 in determining a location of each of the tracking markers 120 (e.g., relative to each other, relative to a predetermined coordinate system, and/or relative to one or more components of the system 100 (e.g., an imaging device 104 and/or 108) or similar components). For example, the indicia may comprise a visual indicator that allows the imaging devices 104 and/or 108 (and/or a processor associated with the imaging devices 104 and/or 108, such as a processor 204) to determine a location of each of the tracking markers 120 relative to the imaging devices 104 and/or 108. Additionally or alternatively, the indicia may assist one or more components of the system 100 in identifying the tracking markers 120. For example, the tracking markers may include light emitting diodes (LEDs) that assist one or more components of the system in identifying each tracking marker 120 and in distinguishing the tracking markers 120 from the mesh 116 and other surroundings. In embodiments where the imaging devices 104 and/or 108 capture an image (e.g., image data and/or image information) of the tracking markers 120, the indicia provided by the tracking markers 120 may permit one or more components of the system 100 or similar components (e.g., computing device 202, robotic arm 112, etc.) to determine the location (e.g., pose, position, orientation, etc.) of the tracking markers 120 (e.g., position of each of the tracking markers 120 relative to any one or more components of the system 100). The system 100 (or components thereof) may use the location information of the tracking markers 120 to determine a work volume (e.g., work volume boundary, virtual surface, etc.), as further described below.

The indicia provided by the tracking markers 120 may be passively and/or actively generated by the tracking markers 120. For example, the tracking markers 120 may comprise or provide a passive indication that may be independent of the components of the system 100 or similar components (e.g., the tracking markers 120 may simply reflect radiation or other electromagnetic waves, which reflections may be detected by the imaging devices 104, 108 and/or other sensors of the system 100, and/or the tracking markers 120 may be color-coded). In some embodiments, the tracking markers 120 may utilize an active indication that can be manipulated by one or more components of the system 100 or similar components (e.g., a signal indication such as an RF signal, with each of the tracking markers 120 producing an RF signal dependent upon the individual tracking marker, one or more signals sent from a component or components of the system 100, combinations thereof, and/or the like).

The indicia may vary between each of the tracking markers 120 in a variety of aspects. For example, the indicia may vary in frequency, intensity, and/or pulse rate. For instance, a color used as visual indication on each of the tracking markers 120 may vary in its intensity of color, the amount of color displayed, and any pattern associated therewith (e.g., dots, stripes, dashes, combinations thereof, etc.). Where the tracking markers 120 displaying the colors are LEDs, the tracking markers 120 may also flash, pulsate, or otherwise switch between on and off states at unique rates (relative to each other). In some embodiments, more than one indication may be used to distinguish one or more of the tracking markers 120, and/or combinations of indicia that implement passive and active generations (e.g., tracking markers that output RF signals and contain visual indicia of colors) may be used to distinguish one or more of the tracking markers 120.

The tracking markers 120 may be used by one or more components of a system 100 (e.g., a computing device 202) to determine a work volume and/or a boundary thereof. For example, the imaging devices 104, 108 may capture image data about the mesh 116 from their respective poses, which image data may be analyzed and used to define a work volume boundary through which a robotic arm 112 can and/or cannot move during a surgery or surgical procedure). More particularly, the tracking markers 120 may be used to define a surface that constitutes a work volume boundary 308. The work volume boundary 308, in turn, separates a work volume in which the robotic arm 112 (including a medical device or surgical tool held by the robotic arm) may safely move from a non-work volume or “no-fly zone” in which the robotic arm 112 must move with care or cannot safely move. The work volume boundary 308 may include a perimeter, border, or other outermost boundary to which, but not through which, a robot (e.g., a robotic arm 112) may move during a surgery or surgical procedure. The work volume boundary 308 may be determined using any of the methods mentioned herein.

Once determined, the work volume boundary 308 may be used by a robotic control system to prevent the robotic arm 112 from moving outside of a bounded work volume. For example, the robotic control system may be configured to calculate or otherwise generate movement instructions for the robotic arm 112 based on the work volume boundary 308, and/or to stop the robotic arm 112 from passing through the work volume boundary 308. Additionally or alternatively, the navigation system 236 may track a position of the robotic arm 112 (and/or of an end effector secured to the robotic arm 112) based on a tracking marker affixed thereto, and the navigation system 236 may generate an audible, visible, electronic, or other signal if it detects that the robotic arm 112 is on a trajectory that will result in the robotic arm 112 breaching the work volume boundary 308. In still other embodiments, the robotic arm may be equipped with sensors that detect movement of the robotic arm within a threshold distance from the work volume boundary 308, which in turn may result in generation of a signal that disables and/or prevents the robotic arm from continuing to move toward the work volume boundary 308. One or more components of the system 100 (e.g., the computing device 202, navigation system 236, combinations thereof, etc.) or similar components may be used to assist the maneuvering of the robot and/or the prevention of the robot from moving beyond the work volume boundary 308.

In some embodiments, the tracking markers 120 may be placed in one or more receptacles (e.g., containers, enclosures, etc.) of the mesh 116. The receptacles may be partially or fully embedded within the mesh 116 and may be configured to house each tracking marker of the tracking markers 120. In some embodiments, the receptacles may be openable to allow for storage and/or removal of each of the tracking markers 120. The receptacles may be configured to permit the tracking markers 120 to provide indicia to the system 100. For example, the receptacles may be clear (e.g., partially or completely transparent) in embodiments where the tracking markers 120 provide one or more visual indicia to the system 100. In this example, the transparency may allow one or more components of the system 100 (e.g., imaging device 104, 108) to capture image data associated with the tracking markers 120 while maintaining the tracking markers 120 secure inside the respective receptacles. As a further example, in embodiments where the tracking markers 120 provide RF signals as a form of indicia, the receptacles may be configured to allow the RF signals to be transmitted to one or more components of the system 100 (e.g., to the navigation system 236, the computing device 202, etc.). In some embodiments, the receptacles may be configured to accommodate tracking markers of a spherical or other shape. In some embodiments, the receptacles may be configured to remain closed (e.g., to prevent removal of each of the tracking markers 120). In such embodiments, the tracking markers 120 may be injected into a respective receptacle. The receptacles may be made of various materials, such as a plastic, that may be resilient to physical damage (e.g., resilient to damage caused by the receptacle falling on the floor, being physically impacted, a sterilization process, etc.).

In some embodiments, a subset of the one or more tracking markers 120 (e.g., one or more tracking markers 320) may contain one or more characteristics common to each other but unique relative to the remaining tracking markers 120 on the mesh 116. The one or more characteristics may distinguish (e.g., physically, digitally, visually, etc.) the tracking markers 320 from the other tracking markers 120 on the mesh 116. In some embodiments, the tracking markers may be free reflective spheres and/or mirrored balls. The one or more characteristics of the tracking markers 320 may provide additional and/or alternative information to the system 100. For instance, a subset of tracking markers 320 positioned at locations where a robot (e.g., a robotic arm 112) and/or a surgical tool secured to a robotic arm 112 may be permitted to pass through the work volume boundary 308 may be given or otherwise configured with one or more common characteristics that are unique relative to the remaining tracking markers 120. In some embodiments, tracking markers 320 may define a workspace 324 within the perimeter of the work volume boundary 308, within which a robotic arm 112 (and/or a tool held thereby) may be maneuvered. The workspace 324 may be determined by the system 100 based on the one or more characteristic of the tracking markers 320. The workspace 324 may be a portion or section (e.g., a two-dimensional area or a three-dimensional volume) of the work volume boundary 308 (or corresponding volume).

As noted above, the workspace 324 may indicate a portion of the work volume boundary 308 where a medical device and/or a surgically operable tool (held, for example, by a robotic arm 112) may cross through the work volume boundary 308 into what would otherwise be a “no-fly zone” on the other side of the work volume boundary 308. In various embodiments, the workspace 324 may be or comprise more or less of the work volume boundary 308. In some embodiments, the workspace 324 may be discontinuous (e.g., multiple isolated locations along the first surface 304 of the mesh 116) and may additionally or alternatively mark locations where the robotic arm 112 may pass through (e.g., pierce through, cut through, etc.) the mesh 116. In such embodiments, the workspace 324 may indicate a target surgical site and may allow the robotic arm 112 to be maneuvered to perform a surgical procedure or surgical task (e.g., drilling, cutting, etc.) only within the workspace 324.

In some embodiments, the one or more tracking markers 320 may function as fiducials for registration. More specifically, the imaging device 104 and/or the imaging device 108 may comprise one or more X-ray imaging devices, which may be used to register a patient coordinate space to a robotic coordinate space. The spacing (e.g., horizontal and vertical distance) between each of the one or more tracking markers 120 may be known by the system 100 and/or components thereof. Also in some embodiments, the one or more tracking markers 120 may also operate as optical tracking markers, such that the system 100 and/or components are able to determine a working volume and complete a registration simultaneously. For example, the one or more tracking markers 120 may be arranged in a pre-determined pattern. The system 100 and/or components thereof (e.g., computing device 202) may use spacing information about the tracking markers 120 along with a known coordinate system for a robotic arm (e.g., a robotic arm 112) to register the robotic arm to a patient space while also determining a work volume boundary.

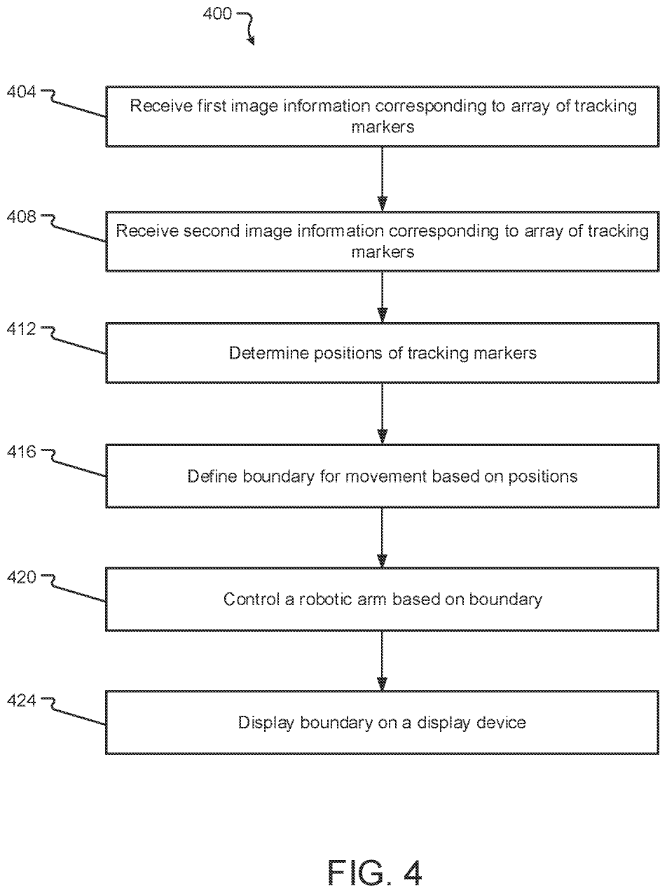

Turning now to FIG. 4, a method 400 for determining a work volume boundary and/or a work volume (for example, in preparation for or during a surgery or other surgical procedure) is shown. The method 400 may utilize one or more components of a system 100 or similar components. The method 400 (and/or one or more steps thereof) may be carried out or otherwise performed, for example, by at least one processor. The at least one processor may be the same as or similar to the processor(s) 204 of the computing device 202 described above. The at least one processor may be part of a robot (such as a robot comprising the robotic arm 112) or part of a navigation system (such as a navigation system 236). A processor other than any processor described herein may also be used to execute the method 400. The at least one processor may perform the method 400 by executing instructions (such as the instructions 224) stored in a memory such as the memory 216. One or more aspects of the method 400 may be performed by or with a surgical robotic arm (e.g., a robotic arm 112) and/or components thereof, a surgeon, or a combination of both using one or more imaging devices (e.g., imaging devices 104, 108) and tracking markers (e.g., a plurality of tracking markers 120 attached to a mesh 116).

The method 400 comprises receiving a first set of image data corresponding to an image (step 404). In some embodiments, the image data corresponds to a single 2D or 3D image. In other embodiments, the image data corresponds to a plurality of 2D or 3D images. The image data may be captured, for example, by an imaging device 104. The image data may be received by, for example, a computing device (e.g., the imaging device 104 may transmit the image data to the computing device 202) and, more specifically, by a processor such as the processor 204 of the computing device 202 or a different processor. The image data may be received, for example, via a communication interface such as the communication interface 208, and/or via a cloud or other network such as the cloud 232. The image data depicts a plurality of tracking markers such as the tracking markers 120 or other tracking devices, which are affixed to (e.g., mounted, attached, glued on, secured to, held within, etc.) a mesh such as the mesh 116. The mesh may be a sterile drape, a flexible sheet, a blanket, or a net configured to be draped or placed over a surgical site for a surgery or surgical procedure. In some embodiments, the tracking markers (e.g., elements affixed to the mesh) may be dispersed along a first surface of the mesh. In some embodiments, the tracking markers may form an array. In some embodiments, the captured image data may depict the array of tracking markers and may be captured by an imaging device placed in a first pose. In other words, the imaging device may be positioned at a location and orientation (e.g., at a first pose 102A) such that the imaging device can view the array of tracking markers. In some embodiments, the method 400 may include storing/saving the image data (e.g., in a database 220, the memory 216, or elsewhere).

The method 400 also comprises receiving a second set of image data corresponding to an image (step 408). In some embodiments, the image data corresponds to a single 2D or 3D image. In other embodiments, the image data corresponds to a plurality of 2D or 3D images. The image data may be captured, for example, by an imaging device other than the imaging device used to capture the first set of image data (e.g., by an imaging device 108), or by the same imaging device but from a different pose. The image data may be received by, for example, a computing device (e.g., the imaging device 108 may transmit the image data to the computing device 202) and, more specifically, by a processor such as the processor 204 of the computing device 202 or a different processor. The image data may be received, for example, via a communication interface such as the communication interface 208, and/or via a cloud or other network such as the cloud 232. The image data depicts the plurality of tracking markers. In other words, the imaging device may be positioned at a location and orientation other than the first pose 102A (e.g., at a second location 102B) such that the imaging device can view the array of tracking markers. In some embodiments, the method 400 may include storing/saving the image data (e.g., in a database 220, the memory 216, or elsewhere). The second set of image data comprises different information than the first set of image data, because the imaging device capturing the second set of image data may be positioned differently with respect to the tracking markers than the imaging device capturing the first set of image data. In some embodiments, the first and second sets of image data are captured simultaneously.

The method 400 includes determining a position associated with the tracking markers (step 412). The tracking markers may be, for example, the plurality of tracking markers 120. The position of the tracking markers may be determined by one or more components of the system 100 (e.g., by the computing device 202, and more specifically by the processor 204). For example, a computing device may receive the first set of image data from one imaging device and the second set of data from the other imaging device and may process both sets of image data. In some embodiments, the computing device may combine the first and second image data to determine a location of the tracking markers relative to a predetermined coordinate system, a robotic arm, and/or other components (e.g., other components of the system 100).

In some embodiments, the computing device may utilize one or more indicia generated by the tracking markers to facilitate determination of the position of each of the tracking markers, and/or to distinguish one or more tracking markers from one or more other tracking markers. For example, each tracking marker in the plurality of tracking markers may comprise a passive and/or active indication (e.g., a color and an RF signal, respectively) that the computing device may use to identify each individual tracking marker.

The method 400 also comprises defining a boundary for movement based on the positions of the tracking markers (step 416). The boundary may correspond to or be represented by, for example, a virtual surface (in a robotic, navigation, or other coordinate space) that comprises, connects, and/or otherwise includes points corresponding to the determined position of the plurality of tracking markers. The boundary may be, for example, a work volume boundary 308. In some embodiments, the defining the boundary may comprise taking into account any additional or alternative tracking markers (e.g., a plurality of tracking makers 320) which may define different boundary conditions for movement of a robotic arm or otherwise. In such embodiments, the computing device may define additional or alternative boundaries (e.g., a workspace 324) that may increase, restrict, change, or otherwise alter a working volume for the robotic arm.

In some embodiments, the step 416 also comprises determining a work volume based on the boundary. The work volume may be, for example, a volume above the boundary (e.g., on an opposite side of the boundary from the patient). In some embodiments, the work volume may extend through the boundary, but only at one or more positions defined by unique tracking markers such as the tracking markers 320. The step 416 may also comprise determining a “no-fly zone” based on the boundary. The no-fly zone may be, for example, a volume below the boundary (e.g., on the same side of the boundary as the patient).

The method 400 also comprises controlling a robotic arm based on the defined boundary (step 420). The robotic arm may be, for example, a robotic arm 112. The robotic arm may be manipulated based on the defined movement boundaries (e.g., a work volume boundary such as the boundary 308, one or more workspaces such as the workspace 324, combinations thereof, and/or the like). In some embodiments, the robotic arm may be manipulated to avoid certain areas (e.g., any area on the same side of the work volume boundary as the patient, unless in a workspace) and may be configured to be capable of being maneuvered and/or being configured to perform certain unique movements in other areas (e.g., workspace 324) of the work volume. Where the step 416 comprises determining a work volume based on the boundary, the step 420 may comprise controlling the robotic arm based on the work volume.

The method 400 also comprises causing the determined boundary to be displayed on a display device (step 424). The display device may be, for example, a user interface 212, and may be capable of rendering a visual depiction of the determined boundary and/or a corresponding work volume such that it may be viewed by a user (e.g., a surgeon). The rendering of the boundary may allow the user to better understand the boundary and, in embodiments where the robotic arm is at least partially controlled by the user, to better direct the robotic arm. In some embodiments, the display device may display the detected position of the plurality of tracking markers along with the work volume defined thereby (e.g., so that a surgeon or other user can verify the accuracy of the determined boundary). In such embodiments, the display device may display the tracking markers with different visual indicia based on the type of tracking marker. For instance, the display device may display each of the tracking markers differently based on any active and/or passive indicia associated therewith. In some embodiments, the display device may display metadata associated with each of the plurality of tracking markers, which may assist a user (e.g., a surgeon) to distinguish the tracking markers on the display device and thus to better view the boundary and/or an associated work volume on the display device.

In some embodiments, the virtual surface may be updated with additional markers (e.g., virtual markers) after the boundary is defined. The additional markers may be displayed on the display device. In such embodiments, the additional markers may be added automatically by one or more components of the system (e.g., a computer device 102), by a user (e.g., a surgeon), and/or combination thereof. The additional markers may be added for a variety of reasons, such as to identify one or more critical locations on the work volume (e.g., portions of the work volume boundary through which the robotic arm may pass), to highlight portions of the working volume boundary that correspond to one or more surgical tasks, to update the work volume boundary based on a result of the procedure or a task thereof, to adjust the boundary to reflect a newly added tool or other medical equipment, or to reflect feedback of one or more sensors (e.g., sensors attached to a robotic arm 112), and/or for any other reason.

The present disclosure encompasses embodiments of the method 400 that comprise more or fewer steps than those described above, and/or one or more steps that are different than the steps described above.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for determining a work volume, the method comprising:
receiving, from an imaging device, image information corresponding to an array of tracking markers fixed to a flexible mesh, the mesh placed over a patient and over at least one surgical instrument adjacent to or connected to the patient;
determining, based on the image information, a position of each tracking marker in the array of tracking markers;
defining a boundary for movement of a robotic arm based on the determined tracking marker positions, such that the robotic arm does not contact the patient or the at least one surgical instrument during movement of the robotic arm; and
controlling the robotic arm based on the defined boundary.

2. The method of claim 1, wherein each tracking marker of the array of tracking markers is secured to the flexible mesh with an adhesive.

3. The method of claim 2, wherein each tracking marker of the array of tracking markers is a reflective sphere.

4. The method of claim 1, wherein each tracking marker of the array of tracking markers is an infrared emitting diode (IRED).

5. The method of claim 1, wherein at least one of the array of tracking markers comprises a selectively adjustable parameter, and wherein the selectively adjustable parameter is one of color, intensity, or frequency.

6. The method of claim 1, wherein a subset of tracking markers in the array of tracking markers comprises a unique characteristic relative to a remainder of tracking markers in the array of tracking markers, the unique characteristic indicative of a location at which the robotic arm may pass through the defined boundary.

7. The method of claim 1, wherein the method further comprises:
determining, based on the image information, an orientation of each tracking marker in the array of tracking markers.

8. The method of claim 1, wherein the flexible mesh substantially conforms to the patient and the at least one surgical instrument.

9. The method of claim 8, wherein the flexible mesh remains within three inches of an underlying surface of the patient or the at least one surgical instrument.

10. A system, comprising:
a processor; and
a memory storing instructions for execution by the processor that, when executed by the processor, cause the processor to:
receive, from a first imaging device in a first pose, first image information corresponding to a plurality of tracking devices flexibly connected to each other;
receive, from a second imaging device in a second pose different than the first pose, second image information corresponding to the plurality of tracking devices;
determine, based on the first image information and the second image information, a position of each tracking device in the plurality of tracking devices;
define a work volume boundary based on the determined tracking device positions; and
control a robotic arm based on the work volume boundary.

11. The system of claim 10, wherein the plurality of tracking devices is uniformly distributed across a first surface of a flexible drape, the flexible drape flexibly connecting the tracking devices to each other.

12. The system of claim 10, wherein each tracking device of the plurality of tracking devices is glued to the flexible drape.

13. The system of claim 10, wherein each tracking device of the plurality of tracking devices is physically secured within a net that flexibly connects the tracking devices to each other.

14. The system of claim 10, wherein a flexible sheet flexibly connects the plurality of tracking devices to each other, the flexible sheet comprising a plurality of receptacles, each receptacle configured to hold one of the plurality of tracking devices.

15. The system of claim 14, wherein each of the plurality of receptacles is a plastic sphere, and wherein each of the plastic spheres is injected with an IRED.

16. The system of claim 10, wherein the defined work volume boundary separates a first volumetric section from a second volumetric section, wherein the processor causes the robotic arm to move within the first volumetric section, and wherein the processor prevents the robotic arm from maneuvering within the second volumetric section.

17. The system of claim 10, wherein the plurality of tracking devices is draped over a surgical site.

18. The system of claim 10, wherein the memory stores additional instructions for execution by the processor that, when executed, further cause the processor to:
cause a visual representation of the defined work volume boundary to be displayed on a display device.

19. A system, comprising:
a processor;
a first imaging device positioned in a first location and in communication with the processor;
a blanket comprising a plurality of tracking markers arranged thereon;
a robotic arm; and
a memory storing instructions for execution by the processor that, when executed by the processor, cause the processor to:
receive, from the first imaging device, first image information corresponding to the plurality of tracking markers;
determine, based on the first image information, a position of each tracking marker of the plurality of tracking markers;
define a virtual surface based on the determined tracking marker positions; and
control the robotic arm based on the defined virtual surface.

20. The system of claim 19, wherein the memory stores additional instructions for execution by the processor that, when executed, further cause the processor to:
receive, from a second imaging device positioned in a second location different from the first location and in communication with the processor, second image information corresponding to the plurality of tracking markers, wherein the position of each tracking marker of the plurality of tracking markers is determined using the second image information.
